(19) United States
(12) Patent Application Publication
(10) Pub. No.: US 2024/0344126 A1
(43) Pub. Date: Oct. 17, 2024

(54) COMPOSITIONS, SYSTEMS, AND METHODS FOR DETERMINATION OF T2R AND/OR T1R PHENOTYPE IN INDIVIDUALS TO PREDICT WINE TASTING PREFERENCES, CHARACTERIZATION OF WINE IN RELATION TO INDIVIDUAL TASTING PREFERENCES, AND SELECTION OF WINE

(71) Applicant: Phenomune, LLC, The Woodlands, TX (US)

(72) Inventor: Henry P. Barham, Baton Rouge, LA (US)

(73) Assignee: Phenomune, LLC, The Woodlands, TX (US)

(21) Appl. No.: 18/513,534

(22) Filed: Nov. 18, 2023

Related U.S. Application Data
(63) Continuation-in-part of application No. 17/693,928, filed on Mar. 14, 2022.
Application No. 17/693,928 is a continuation-in-part of application No. 17/500,755, filed on Oct. 13, 2021, now abandoned.
(60) Provisional application No. 63/384,363, filed on Nov. 18, 2022.
(60) Provisional application No. 63/350,397, filed on Jun. 8, 2022.
(60) Provisional application No. 63/340,630, filed on May 11, 2022.
(60) Provisional application No. 63/092,279, filed on Oct. 15, 2020.

(51) Int. Cl. C12Q 1/6876; G01N 21/77; G01N 33/50; G01N 33/566

(52) U.S. Cl. CPC C12Q 1/6876; G01N 33/5041; G01N 33/566; C12Q 2600/158; G01N 2021/7769; G01N 2333/726

(57) ABSTRACT

A method of measuring phenotypic expression of T2Rs and/or TIRs for a subject to predict taste preferences for wines may include stimulating T2Rs and/or TIRs of a subject with agonists and detecting products released as a result of stimulation of the T2Rs and/or T1Rs. A method may include recording discerned levels of taste perception after stimulation with agonists and correlating the discerned level to phenotypic expression, which may be used to predict taste preferences for wine profiles. A method may include calculating wine bin scores for predicted wine preferences, from phenotypic expression of T2Rs and/or T1Rs, obtaining taste preference information, and determining the subject's taste preference scores for wines administered to the subject.

### CROSS-REFERENCE TO RELATED APPLICATIONS

This application is related to each of the following: U.S. Provisional Application No. 63/384,363, filed on Nov. 11, 2022, titled COMPOSITIONS, SYSTEMS, AND METHODS FOR DETERMINATION OF T2R AND/OR TIR PHENOTYPE IN INDIVIDUALS TO PREDICT WINE TASTING PREFERENCES, CHARACTERIZATION OF WINE IN RELATION TO INDIVIDUAL TASTING PREFERENCES, AND SELECTION OF WINE, which is incorporated herein by reference in entirety; U.S. Provisional Application No. 63/350,397, filed Jun. 8, 2022, titled COMPOSITIONS, SYSTEMS, AND METHODS FOR DETERMINATION OF T2R AND/OR TIR PHENOTYPE IN INDIVIDUALS TO PREDICT WINE TASTING PREFERENCES, CHARACTERIZATION OF WINE IN RELATION TO INDIVIDUAL TASTING PREFERENCES, AND SELECTION OF WINE, which is incorporated herein by reference in entirety; U.S. Provisional Application No. 63/340,630, filed May 11, 2022, titled COMPOSITIONS, SYSTEMS, AND METHODS FOR TREATMENT WITH T2R AGONIST NASAL SPRAYS AND APPLICATIONS THEREOF, which is incorporated herein by reference in entirety; U.S. Non-provisional application Ser. No. 17/693,928 filed Mar. 14, 2022, titled TESTING METHODS FOR DETERMINATION OF T2R PHENOTYPE AND APPLICATIONS THEREOF, which is incorporated herein by reference in entirety; PCT Application No. PCT/US22/24892 filed Apr. 14, 2022, titled TESTING METHODS FOR DETERMINATION OF T2R PHENOTYPE AND APPLICATIONS THEREOF, which is incorporated herein by reference in entirety; U.S. Non-provisional application Ser. No. 17/500,755 filed Oct. 13, 2021, titled TESTING METHODS FOR DETERMINATION OF T2R PHENOTYPE AND APPLICATIONS THEREOF, which is incorporated herein by reference in entirety; PCT Application No. PCT/US21/54810 filed, on Oct. 13, 2021, titled TESTING METHODS FOR DETERMINATION OF T2R PHENOTYPE AND APPLICATIONS THEREOF, which is incorporated herein by reference in entirety; and U.S. Provisional Application No. 63/092,279, filed Oct. 15, 2020, titled TESTING METHODS FOR DETERMINATION OF T2R PHENOTYPE AND APPLICATIONS THEREOF, which is incorporated herein by reference in entirety.

### FIELD OF THE INVENTION

The disclosure relates to the characterization of chemosensory receptors, stimulation of chemosensory receptors, and applications of stimulating chemosensory receptors. More particularly, the disclosure relates to the characterization and stimulation of T2Rs and/or T1Rs and applications of stimulating T2Rs and/or T1Rs. The disclosure relates to compositions, systems, and methods for determination of T2R and/or T1R phenotype expression in individuals to predict or characterize individual wine tasting preferences, characterization of wine in relation to individual taste preferences arising from T2R and/or T1R phenotype expression in individuals, and selection of wine in relation to predicted or characterized individual wine tasting preferences arising from T2R and/or T1R phenotype expression in individuals, which may be determined by applications of compositions, systems, and methods as herein disclosed.

### BACKGROUND OF THE INVENTION

Chemosensory receptors may be encoded by six families of genes including trace amine-associated receptors (TAAR), olfactory receptors (OR), vomeronasal receptor type 1 and 2 (V1R and V2R), and taste receptors type 1 and 2 (T1R and T2R). All the chemosensory receptor proteins may be G-protein coupled receptors. Chemosensory receptors may be expressed on the surface of solitary chemosensory cells (SCCs). Receptors belonging to Taste Receptor Family-I subtype 2 and 3 (T1R2/T1R3) detect sweet compounds such as glucose and sucrose. Taste Receptor Family-2 receptors (T2Rs) detect bitter taste compounds. Greater than fifty (50) T2Rs have been characterized. Stimulation of T2Rs activates, at least, the canonical taste signaling cascade involving phospholipase C 2 (PLC 2) and transient receptor potential cation channel subfamily M member 5 [Nei, M., Niimura, Y. & Nozawa, M. The evolution of animal chemosensory receptor gene repertoires: roles of chance and necessity. Nature Reviews Genetics 9, 951-963 (2008]. The aforementioned manuscript is hereby incorporated by reference in its entirety. Unless expressly stated to the contrary, the form of instantiation of any element of this disclosure shall be considered as being only a matter of design choice.

T2Rs may be genetically diverse, a phenomenon that helps to explain the wide variety of taste preferences both within and between cultures. Many individuals find various bitter foods to be detestable, while others do not have the same aversive response. This genetic variation of T2Rs may be found on the tongue, but not exclusively. More recently, bitter and sweet receptors have been discovered in a variety of extra-oral tissues including the brain, thyroid, pancreas, testes, and throughout the respiratory and gastrointestinal tracts. The activity of T2Rs and/or T1Rs may be useful in predicting taste preferences. There is a need for improved compositions, systems, and methods for the determination of T2R and/or T1R phenotype expression in individuals to predict or characterize individual wine-tasting preferences. There is a need for improved compositions, systems, and methods for the determination of T2R and/or T1R phenotype expression in individuals to characterize wine in relation to individual taste preferences arising from T2R and/or T1R phenotype expression in individuals. There is a need for characterization and selection of wine in relation to predicted or characterized individual wine-tasting preferences arising from T2R and/or T1R phenotype expression in individuals, which may be determined by applications of improved compositions, systems, and methods as herein disclosed.

The above-mentioned shortcomings, disadvantages, and problems may be addressed herein, as will be understood by those skilled in the art upon reading and studying the following specifications. This summary is provided to introduce a selection of concepts in a simplified form that may be further described below in more detail in the Detailed Description. This summary is not intended to identify key or essential features of the claimed subject matter. Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

### BRIEF SUMMARY OF THE INVENTION

The need exists for reliable methods of predicting taste preferences of wines. Phenotypic expression of T2Rs and/or T1 Rs may correlate to taste preferences of different types of wine.

In embodiments, a method for predicting the wine preferences of a subject may be provided, the method comprising: first determining phenotypic expression of T2Rs and/or T1Rs, wherein the determining may be from an actual test administered to detect the functionality of T2Rs and/or T1Rs; obtaining taste preference information from the subject; second determining the subject's taste preference scores for one or more wines administered to the subject; predicting wine preferences for the subject.

In embodiments, a method for predicting the wine preferences of a subject may be provided, the method comprising: first determining phenotypic expression of T2Rs and/or Tl Rs, wherein the determining may be from an actual test administered to detect the functionality of T2Rs and/or T1Rs; second determining the subject's taste preference scores for one or more wines administered to the subject; predicting wine preferences for the subject.

In embodiments, a method for predicting the wine preferences of a subject may be provided, the method comprising: first determining the phenotypic expression of T2Rs and/or T1 Rs, wherein the determining may be from an actual test administered to detect the functionality of T2Rs and/or T1Rs; predicting wine preferences for the subject.

Apparatus, systems, and methods of varying scope may be described herein. These aspects may be indicative of various non-limiting ways in which the disclosed subject matter may be utilized, all of which may be intended to be within the scope of the disclosed subject matter. In addition to the aspects and advantages described in this summary, further aspects, features, and advantages will become apparent by reference to the associated drawings, detailed descriptions, and claims.

### BRIEF DESCRIPTION OF THE DRAWINGS

The novel features believed characteristic of the disclosed subject matter will be set forth in any claims that are filed later. The disclosed subject matter itself, however, as well as a preferred mode of use, further objectives, and advantages thereof, will best be understood by reference to the following detailed description of an illustrative embodiment when read in conjunction with the accompanying drawings, wherein:

FIG. 1 is a simplified flow diagram illustrating aspects of a method 100 of treatment in an embodiment;

FIG. 2 is a simplified flow diagram illustrating aspects of method 200 of treatment in an embodiment;

FIG. 3 is a simplified flow diagram illustrating aspects of method 300 of treatment in an embodiment.

### DETAILED DESCRIPTION OF ILLUSTRATIVE EMBODIMENT

In this detailed description, reference is made to the accompanying drawings which form a part hereof, and in which is shown by way of illustration specific an embodiment which may be practiced. Reference now should be made to the drawings, in which the same reference numbers are used throughout the different figures to designate the same components. These embodiments are described in sufficient detail to enable those skilled in the art to practice the embodiment and disclosure. It is to be understood that other embodiments may be utilized, and that logical, mechanical, electrical, and other changes may be made without departing from the scope of the embodiment and disclosure. In view of the foregoing, the following detailed description is not to be taken as limiting the scope of the an embodiment or disclosure.

The terminology used herein is for the purpose of describing a particular embodiment only and is not intended to be limiting. As used herein, the singular forms “a”, “an”, and “the” are intended to include the plural forms as well, unless the context clearly indicates otherwise. It will be further understood that the terms “comprises” and/or “comprising” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

It will be appreciated that for simplicity and clarity of illustration, where considered appropriate, reference numerals may be repeated among the figures to indicate corresponding or analogous elements. In addition, numerous specific details are set forth in order to provide a thorough understanding of the implementations described herein. However, it will be understood by those of ordinary skill in the art that the implementations described herein may be practiced without these specific details. In other instances, well-known methods, procedures, and components have not been described in detail so as not to obscure the implementations described herein. Also, the description is not to be considered as limiting the scope of the implementations described herein.

The detailed description set forth herein in connection with the appended drawings is intended as a description of exemplary an embodiment in which the presently disclosed apparatus and system can be practiced. The term “exemplary” used throughout this description means “serving as an example, instance, or illustration,” and should not necessarily be construed as preferred or advantageous over other an embodiment.

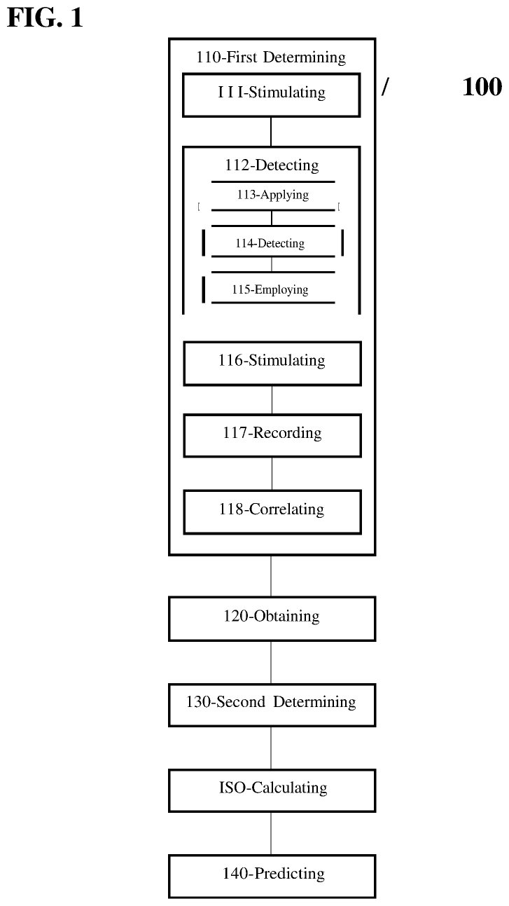

Illustrated in FIG. 1 is a simplified flow diagram of Method 100 of a method for predicting the wine preferences of a subject. The method may comprise first determining 110 phenotypic expressions of T2Rs and/or T1 Rs, wherein the determining may be from an actual test administered to detect the functionality of T2Rs and/or T1 Rs. The method may further comprise obtaining 120 taste preference information from the subject. The method may further comprise a second determining 130 of the subject's taste preference scores for one or more wines administered to the subject. The test method may further comprise predicting 140 wine preferences for the subject.

Referring to FIG. 1, method 100 may further include calculating 150 wine bin scores based on the first determining phenotypic expression, obtaining taste preference information, and the second determining the subject's taste preference scores. The wine bins may comprise one or more wine bins selected from the group consisting of a first wine bin for sweet white wines, a second wine bin for crisp and citrusy white wines, a third wine bin for crisp and floral white wines, a fourth wine bin for unoaked full-bodied white wines, a fifth wine bin for oaked full-bodied white wines, a sixth wine bin for light cherry red wines, a seventh wine bin for light chocolaty raspberry red wines, and an eighth wine bin for bold smoky blackberry red wines. The wine bins may comprise one or more wine bins selected from the group consisting of a first wine bin for bitter and/or sweet wines, a second wine bin consisting of bland choices both away from sweet and bitter, and a third wine bin consisting of a broad range of wine choices.

Referring to FIG. 1, method 100 may further include the wine bin scores used to predict that the subject will prefer wines from a wine bin having a higher wine bin score than a wine bin having a lower wine bin score.

Referring to FIG. 1, the Method 100 may further include the first determining 110 may comprise stimulating 111 T2Rs and/or T1Rs of a human subject with one or more agonists, and detecting 112 one or more products released as a result of stimulation of the T2Rs and/or T1 Rs, wherein the detecting of the products comprises a method comprising either:
A-i) applying 113 one or more reagents which interact with one or more of the products released because of the stimulation of the T2Rs and/or T1 Rs, and
A-ii) detecting 114 the interaction of the reagent with one or more products, optionally by employing chemiluminescence, an electrochemical sensor, or an optical sensor, to detect the interaction of one or more reagents with one or more of the products, or
B) employing 115 chemiluminescence, an electrochemical sensor, or an optical sensor to detect one or more of the products released because of the stimulation of the T2Rs and/or T1Rs.

Referring to FIG. 1, the agonists may be separately applied 111 prior to the reagent 113 which interacts with one or more of the products as a result of stimulation of T2Rs and/or T1 Rs.

Referring to FIG. 1, the agonist and the reagent that interacts with one or more products released as a result of stimulation of T2Rs and/or T1Rs may be contained on or the same test medium.

Referring to FIG. 1, the agonists may be selected from the group consisting of caffeine, denatonium, strychnine, quinine, terpenes, phenylthiocarbamide, thiourea, sodium benzoate, and any two or more of the foregoing.

Referring to FIG. 1, the detection of the products may comprise applying a test medium containing the reagents which interact with the products.

Referring to FIG. 1, the reagents which interact with the products released as a result of stimulation of T2Rs and/or T1 Rs may comprise a Griess reagent.

Referring to FIG. 1, the agonist(s) may be selected from caffeine, denatonium, strychnine, quinine, terpenes, phenylthiocarbamide, thiourea, sodium benzoate, and any two or more of the foregoing, and wherein the detecting of the products may comprise applying a test medium containing the reagents which interact with the products. The reagents which interact with the products released as a result of stimulation of T2Rs and/or T1 Rs may comprise a Griess reagent.

Referring to FIG. 1, the first determining may further comprise:
i) stimulating 116 T2Rs and/or T1Rs by exposing at least a portion of tissue, for example, the mouth of the human subject to one or more agonists,
ii) recording 117 a discerned level of taste perception by the human subject after the stimulation, and
iii) correlating 118 the discerned level of taste perception to the level of phenotypic expression of the T2Rs and/or T1 Rs.

Referring to FIG. 1, the agonists for stimulating 116 may be selected from the group consisting of caffeine, denatonium, strychnine, quinine, terpenes, phenylthiocarbamide, thiourea, sodium benzoate, and any two or more of the foregoing.

Referring to FIG. 1, the agonists for stimulating 116 may be selected from the group consisting of caffeine, denatonium, strychnine, quinine, xylitol, grapefruit seed extract or naringenin, a terpene, and any two or more of the foregoing.

Referring to FIG. 1 steps 116 and 117 may be repeated one or more times, and the stimulating 116 by each of one or more different agonists may be sequential, the recording 117 of each discerned level of taste perception by the human subject may occur after each stimulation, and the correlating 118 may be of one or more of the discerned levels of taste perception to the level of phenotypic expression of the T2Rs and/or T1Rs.

Referring to FIG. 1, the agonists for stimulating 116 may be selected from the group consisting of caffeine, denatonium, strychnine, quinine, terpenes, phenylthiocarbamide, thiourea, sodium benzoate, and any two or more of the foregoing.

Referring to FIG. 1, the method may be performed by providing a computing platform that enables the execution of the method, for recommending wines by determining taste perception of an individual and determining wine preferences in relation to taste perception.

Referring to FIG. 1, the method may further comprise formulating and making wines in relation to determining taste perception of an individual by diagnostic tests to establish a taster group and determining wine preferences in relation to taste perception.

Referring to FIG. 1, the method may comprise conducting wine tasting events including the use of diagnostic test kits to determine the taste perception of an individual and determine wine preferences in relation to taste perception for recommending wines in relation to such taste perception.

Referring to FIG. 1, the method may comprise advising winemakers and suppliers in relation to taste perception of consumers, determined in relation to diagnostic tests establishing taster groups and preferences in relation to taste perception.

Referring to FIG. 1, the method may comprise using a test kit configured to enable performing said method, said test kit comprising at least one of said agonists contained in at least one test medium for stimulating 116.

Referring to FIG. 1, the method may comprise using a test kit configured to enable performing said method, said test kit comprising at least one of said agonists to stimulate 111, contained in at least one test medium, a reagent in a test medium to apply 113, said reagent capable of interaction with a product released in relation to said stimulating, said interaction providing a detectable phenomenon.

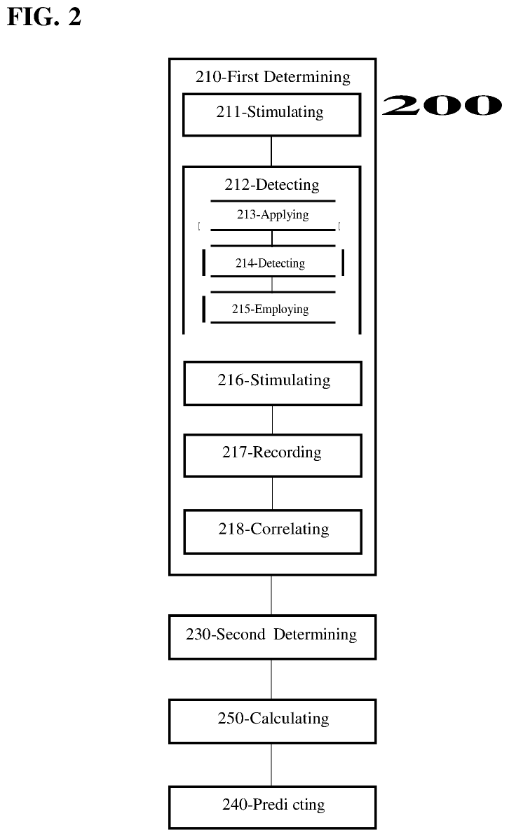

Illustrated in FIG. 2 is a simplified flow diagram of Method 200 of a method for predicting wine preferences of a subject. The method may comprise first determining 210 phenotypic expression of T2Rs and/or T1 Rs, wherein the determining may be from an actual test administered to detect the functionality of T2Rs and/or T1 Rs. The method may also include a second determining 230 the subject's taste preference scores for one or more wines administered to the subject. The method may also include predicting 240 wine preferences for the subject. The method may also further comprise calculating 250 wine bin scores based on the first determining phenotypic expression, and the second determining the subject's taste preference scores.

Referring to FIG. 2, the method may comprise the wine bins comprising one or more wine bins selected from the group consisting of a first wine bin for sweet white wines, a second wine bin for crisp and citrusy white wines, a third wine bin for crisp and floral white wines, a fourth wine bin for unoaked full-bodied white wines, a fifth wine bin for oaked full-bodied white wines, a sixth wine bin for light cherry red wines, a seventh wine bin for light chocolaty raspberry red wines, and an eighth wine bin for bold smoky blackberry red wines.

Referring to FIG. 2, the method may comprise the wine bins comprising one or more wine bins selected from the group consisting of a first wine bin for bitter and/or sweet wines, a second wine bin consisting of bland choices both away from sweet and bitter, and a third wine bin consisting of a broad range of wine choices.

Referring to FIG. 2, the method may comprise the wine bin scores being used to predict that the subject will prefer wines from a wine bin having a higher wine bin score than a wine bin having a lower wine bin score.

Referring to FIG. 2, the method may comprise the first determining 210 further comprising stimulating 211 T2Rs and/or T1Rs of a human subject with one or more agonists and detecting 212 one or more products released as a result of stimulation of the T2Rs and/or T1 Rs, wherein the detecting of the products comprises a method comprising either:
A-i) applying 213 one or more reagents which interact with one or more of the products released because of the stimulation of the T2Rs and/or T1 Rs, and
A-ii) detecting 214 the interaction of the reagent with one or more products, optionally by employing chemiluminescence, an electrochemical sensor, or an optical sensor, to detect the interaction of one or more reagents with one or more of the products, or
B) employing 215 chemiluminescence, an electrochemical sensor, or an optical sensor to detect one or more of the products released because of the stimulation of the T2Rs and/or T1Rs.

Referring to FIG. 2, the method may further comprise the agonists being separately applied prior to the reagent which interacts with one or more of the products as a result of stimulation of T2Rs and/or T1 Rs.

Referring to FIG. 2, the method may comprise the agonist and the reagent that interacts with one or more products released as a result of stimulation of T2Rs and/or T1Rs may be contained on or the same test medium.

Referring to FIG. 2, the method may further comprise the agonists being selected from the group consisting of caffeine, denatonium, strychnine, quinine, terpenes, phenylthiocarbamide, thiourea, sodium benzoate, and any two or more of the foregoing.

Referring to FIG. 2, the method may further comprise the detection of the products comprises applying a test medium containing the reagents which interact with the products.

Referring to FIG. 2, the method may further comprise the reagents which interact with the products released as a result of stimulation of T2Rs and/or T1 Rs comprise a Griess reagent.

Referring to FIG. 2, the method may further comprise the agonist(s) may be selected from caffeine, denatonium, strychnine, quinine, terpenes, phenylthiocarbamide, thiourea, sodium benzoate, and any two or more of the foregoing, and wherein the detecting of the products comprises applying a test medium containing the reagents which interact with the products.

Referring to FIG. 2, the method may further comprise the reagents which interact with the products released as a result of stimulation of T2Rs and/or T1 Rs comprise a Griess reagent.

Referring to FIG. 2, the method may further comprise the first determining 210 further comprises of:
i) stimulating 216 T2Rs and/or T1Rs by exposing at least a portion of tissue, for example, the mouth of the human subject to one or more agonists,
ii) recording 217 a discerned level of taste perception by the human subject after the stimulation, and
iii) correlating 218 the discerned level of taste perception to the level of phenotypic expression of the T2Rs and/or T1 Rs.

Referring to FIG. 2, the method for stimulating 216 may further include the agonists may be selected from the group consisting of caffeine, denatonium, strychnine, quinine, terpenes, phenylthiocarbamide, thiourea, sodium benzoate, and any two or more of the foregoing.

Referring to FIG. 2, the method may further include first determining 210 further comprises wherein the stimulating 216 may comprise agonist being selected from the group consisting of caffeine, denatonium, strychnine, quinine, xylitol, grapefruit seed extract or naringenin, a terpene, and any two or more of the foregoing.

Referring to FIG. 2, the method may further include steps 216 and 217 may be repeated one or more times and the stimulating 216 by each of one or more different agonists may be sequential, the recording 217 of each discerned level of taste perception by the human subject occurs after each stimulation, and the correlating 218 may be of one or more of the discerned levels of taste perception to the level of phenotypic expression of the T2Rs and/or T1Rs.

Referring to FIG. 2, the method may further include the stimulating 216 with at least one agonist which may be selected from the group consisting of caffeine, denatonium, strychnine, quinine, terpenes, phenylthiocarbamide, thiourea, sodium benzoate, and any two or more of the foregoing.

Referring to FIG. 2, the method may further include providing a computing platform that enables the execution of the method, for recommending wines by determining taste perception of an individual and determining wine preferences in relation to taste perception.

Referring to FIG. 2, the method may further include formulating and making wines in relation to determining taste perception of an individual by diagnostic tests to establish a taster group and determining wine preferences in relation to taste perception.

Referring to FIG. 2, the method may further include conducting wine-tasting events including the use of diagnostic test kits to determine the taste perception of an individual and determine wine preferences in relation to taste perception for recommending wines in relation to such taste perception.

Referring to FIG. 2, the method may further include advising winemakers and suppliers in relation to taste perception of consumers, determined in relation to diagnostic tests establishing taster groups and preferences in relation to taste perception.

Referring to FIG. 2, the method may further include using a test kit configured to enable performing said method, said test kit comprising at least one of said agonists contained in at least one test medium.

Referring to FIG. 2, the method may further include a test kit configured to enable performing said method, said test kit comprising at least one of said agonists contained in at least one test medium. The test may further include a reagent in a test medium, said reagent capable of interaction with a product released in relation to said stimulating, said interaction providing a detectable phenomenon.

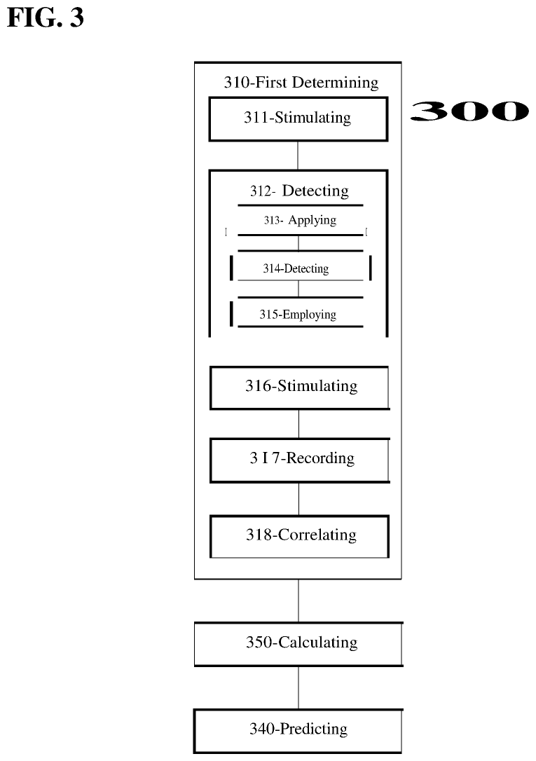

Illustrated in FIG. 3 is a simplified flow diagram of Method 300 of a method for predicting wine preferences of a subject, the method comprising first determining 310 phenotypic expressions of T2Rs and/or T1 Rs, wherein the determining may be from an actual test administered to detect the functionality of T2Rs and/or T1 Rs; predicting 340 wine preferences for the subject.

Referring to FIG. 3, the method may further include calculating 350 wine bin scores based on the first determining phenotypic expression.

Referring to FIG. 3, the method may further include wine bins that may comprise one or more wine bins selected from the group consisting of a first wine bin for sweet white wines, a second wine bin for crisp and citrusy white wines, a third wine bin for crisp and floral white wines, a fourth wine bin for unoaked full-bodied white wines, a fifth wine bin for oaked full-bodied white wines, a sixth wine bin for light cherry red wines, a seventh wine bin for light chocolaty raspberry red wines, and an eighth wine bin for bold smoky blackberry red wines.

Referring to FIG. 3, the method may further include wine bins that may comprise one or more wine bins selected from the group consisting of a first wine bin for bitter and/or sweet wines, a second wine bin consisting of bland choices both away from sweet and bitter, and a third wine bin consisting of a broad range of wine choices.

Referring to FIG. 3, the method may further include wine bin scores that may be used to predict that the subject will prefer wines from a wine bin having a higher wine bin score than a wine bin having a lower wine bin score.

Referring to FIG. 3, the method may further include the first determining 310 further comprising stimulating 311 T2Rs and/or T1Rs of a human subject with one or more agonists and detecting 312 one or more products released as a result of stimulation of the T2Rs and/or T1 Rs, wherein the detecting of the products comprises a method comprising either:
A-i) applying 313 one or more reagents which interact with one or more of the products released because of the stimulation of the T2Rs and/or T1 Rs, and
A-ii) detecting 314 the interaction of the reagent with one or more products, optionally by employing chemiluminescence, an electrochemical sensor, or an optical sensor, to detect the interaction of one or more reagents with one or more of the products, or
B) employing 315 chemiluminescence, an electrochemical sensor, or an optical sensor to detect one or more of the products released because of the stimulation of the T2Rs and/or T1Rs and/or T1Rs.

Referring to FIG. 3, the method may further comprise the agonists that may be separately applied prior to the reagent which interacts with one or more of the products as a result of stimulation of T2Rs and/or T1 Rs and/or T1 Rs.

Referring to FIG. 3, the method may further comprise the agonist and the reagent that interacts with one or more products released as a result of stimulation of T2Rs and/or T1 Rs and/or T1Rs may be contained on or the same test medium.

Referring to FIG. 3, the method may further comprise the agonists being selected from the group consisting of caffeine, denatonium, strychnine, quinine, terpenes, phenylthiocarbamide, thiourea, sodium benzoate, and any two or more of the foregoing.

Referring to FIG. 3, the method may further comprise the detection of 312 of the products comprise applying a test medium containing the reagents that interact with the products.

Referring to FIG. 3, the method may further comprise the reagents that interact with the products released as a result of stimulation of T2Rs and/or T1Rs and/or T1Rs comprise a Griess reagent.

Referring to FIG. 3, the method may further the agonist(s) being selected from caffeine, denatonium, strychnine, quinine, terpenes, phenylthiocarbamide, thiourea, sodium benzoate, and any two or more of the foregoing, and wherein the detecting of the products comprises applying a test medium containing the reagents which interact with the products.

Referring to FIG. 3, the test method may further comprise the reagents which interact with the products released as a result of stimulation of T2Rs and/or T1Rs that comprise a Griess reagent.

Referring to FIG. 3, the method may further comprise the first determining 310 further comprising:
i) stimulating 316 T2Rs and/or T1Rs and/or T1Rs by exposing at least a portion of tissue, for example, the mouth of the human subject to one or more agonists,
ii) recording 317 a discerned level of taste perception by the human subject after the stimulation, and
iii) correlating 318 the discerned level of taste perception to the level of phenotypic expression of the T2Rs and/or T1 Rs and/or T1 Rs.

Referring to FIG. 3, the method may further comprise the agonists being selected from the group consisting of caffeine, denatonium, strychnine, qumme, terpenes, phenylthiocarbamide, thiourea, sodium benzoate, and any two or more of the foregoing.

Referring to FIG. 3, the method may further comprise the first determining 310 further comprises wherein the agonist may be selected from the group consisting of caffeine, denatonium, strychnine, quinine, xylitol, grapefruit seed extract or naringenin, a terpene, and any two or more of the foregoing.

Referring to FIG. 3, the method may further comprise steps 316 and 317 may be repeated one or more times, and the stimulating 316 by each of one or more different agonists may be sequential, the recording 317 of each discerned level of taste perception by the human subject occurs after each stimulation, and the correlating 318 may be of one or more of the discerned levels of taste perception to the level of phenotypic expression of the T2Rs and/or T1 Rs and/or T1Rs.

Referring to FIG. 3, the method may further comprise the agonists may be selected from the group consisting of caffeine, denatonium, strychnine, quinine, terpenes, phenylthiocarbamide, thiourea, sodium benzoate, and any two or more of the foregoing.

Referring to FIG. 3, the method may further include providing a computing platform that enables the execution of the method, for recommending wines by determining taste perception of an individual and determining wine preferences in relation to taste perception.

Referring to FIG. 3, the method may further comprise formulating and making wines in relation to determining the taste perception of an individual by diagnostic tests to establish a taster group and determining wine preferences in relation to taste perception.

Referring to FIG. 3, the method may further comprise conducting wine-tasting events including use of diagnostic test kits to determine the taste perception of an individual and determine wine preferences in relation to taste perception for recommending wines in relation to such taste perception.

Referring to FIG. 3, the method may further comprise advising winemakers and suppliers in relation to taste perception of consumers, determined in relation to diagnostic tests establishing taster groups and preferences in relation to taste perception.

Referring to FIG. 3, the method may further comprise using a test kit configured to enable performing said method, said test kit comprising: at least one of said agonists contained in at least one test medium.

Referring to FIG. 3, the method may further comprise using a test kit configured to enable performing said method, said test kit comprising: at least one of said agonists contained in at least one test medium. The test kit may further comprise a reagent in a test medium, said reagent capable of interaction with a product released in relation to said stimulating, said interaction providing a detectable phenomenon.

G-Protein Coupled Receptors (GPCRs) make up the largest family of human cell surface receptors and are involved in a wide range of physiological functions, such as sight, taste, smell, behavioral, and mood regulation, and regulation of the immune system. Specific GPCRs, such as sweet (T1R) and bitter taste (T2R) receptors, were first identified in taste bud type II cells and function as chemoreceptors that interact with taste stimuli to initiate a signal transmitted to the brain, resulting in taste perception. Prior research has suggested an inverse relationship between the expression of T1R (Sweet) and T2R (Bitter) taste receptors. Sweet (T1R) GPCRs respond to sugars, such as glucose, fructose, and sucrose, and play a central role in the perception of sweet and umami tastes in humans. A low expression of sweet taste receptors (T1Rs) leads an individual to be less sensitive to sugars. A moderate expression of sweet taste receptors (T1Rs) leads an individual to be more sensitive to sugars. High T1R (Sweet Taste Expression) leads to High

Sweetness Perception or a High Ability to Detect Sugars Low T1R (Sweet Taste Expression) leads to Low Sweetness Perception or a Low Ability to Detect Sugars. Relationship to wine includes Higher sugar content of wines to balance tighter perceived bitterness and/or astringency Bitter (T2R) GPCRs respond to bitter compounds, such as plant sesquiterpene lactones, clerodane, strychnine, denatonium, phenylthiocarbamide (PTC), and propylthiouracil (PROP), and play a central role in the perception of bitter tastes in humans. T2Rs are genetically diverse and help to explain the wide variety of taste preferences both within and between cultures. Some individuals find bitter foods and beverages to be detestable, while others do not have an aversive response. High T2R (Bitter Taste Expression) leads to High Bitterness Perception or a High Ability to Detect Bitterness. Low T2R (Bitter Taste Expression) leads to Low Bitterness Perception or a Low Ability to Detect Bitterness. Relationship to Wine includes a wide range of bitterness and/or astringency to balance the increase in perceived sugar content. The genetic locus for T2R38, TAS2R38, has common polymorphisms that tend to segregate together, and each allele is typically inherited one from each parent, resulting in three primary combinations: PAV/PAV, PAV/AVI, and AVI/AVI, where PAV is the functional allele variant, and AVI is the non-functional allele variant. Common Haplotypes include PAV (Functional) and AVI (Non-Functional). Common Genotypes include PAV/PAV, PAV/AVI, and AVI/AVI. Common Phenotypes include: Intense Bitterness, Mild Bitterness, and No Perceived Bitterness.

The following terminology is germane:
Agonist: A chemical that activates a receptor to produce a biological response.
Allele: Any one of the many potentially alternative (variant) forms of a gene.
Astringency: Defined as dryness, tightening, and puckering sensations perceived in the oral cavity during the intake of astringent compounds. A common sensory example used to explain this sensation to a nonscientific audience is the sensation experienced during the uptake of unripe bananas or persimmons. The term astringency, with its origin in Latin, adstringere, meaning “to bind”, can be induced by the binding of alums, acids, alcohols, and tannins.
Bitter Taste Agonists: A substance meant to activate bitter taste receptors.
Bitter Taste Receptors (BTRs): Components of some taste buds that are specifically tuned to detect bitter tastes.
Bitter Taste Sensitivity: The spectrum of bitter taste intensity levels experienced when exposed to bitter taste agonists.
G-Protein Coupled Receptors (GPCRs): GPCRs make up the largest family of human cell surface receptors and are involved in a wide range of physiological functions, such as sight, taste, smell, behavioral, and mood regulation, and regulation of the immune system.
Specific GPCRs, such as sweet (T1R) and bitter taste (T2R) receptors, were first identified in taste bud type II cells and function as chemoreceptors that interact with taste stimuli to initiate a signal transmitted to the brain, resulting in taste perception.
Genotype: An inherited collection of genetic material. This term can also refer to a specific set of genes. Genotypes can only be determined through genetic testing known as genotyping.
Haplotypes: A collection of alleles on a chromosome that are often inherited, together from a single parent.
PAV/AVI: The two most common haplotypes of the T2R38 gene. PAV is the functional variant, whereas AVI is the non-functional variant.
Phenotype: Observable or measurable traits, such as hair color or eye color. Both genotype and environmental factors may influence phenotype. Often, more than one gene influences a specific trait.
Phenotypic Expression: The measurable/observable traits that indicate phenotype. While a genetic test is required to determine genotype, no genetic material is required to determine phenotype.
Phenylthiocarbamide (PTC) and 6-n-Propylthiouracil (PTU): Each is a chemical that mimics the bitter taste of toxic substances. It is regularly used in bitter taste sensitivity testing, as only very low concentrations are needed to trigger an intense reaction in people with high levels of bitter taste sensitivity.
Sweetness: Called Residual Sugar (RS) in wine. Residual sugar is the unfermented grape sugars (fructose and glucose) left over in the wine after fermentation has completed.
T1R Sweet Taste Receptor: GPCRs that respond to sugars, such as glucose, fructose, and sucrose, and play a central role in the perception of sweet and umami tastes in humans. T2R Bitter Taste Receptor: GPCRs that respond to bitter compounds, such as plant sesquiterpene lactones, clerodane, strychnine, denatonium, phenylthiocarbamide (PTC), and propylthiouracil (PROP), and play a central role in the perception of bitter tastes in humans.
Tannins: Naturally occurring polyphenols found in plants, seeds, bark, wood, leaves, and fruit skins. In wines, tannins are found in grape skins and seeds, and they're also found in wooden barrels. Tannins, including grape-derived condensed tannins, produce sensations of astringency in food and drink and form the “structure” or “body” of red wine.

Apparatus, methods, and systems according to embodiments of the disclosure are described. Although specific embodiments are illustrated and described herein, it will be appreciated by those of ordinary skill in the art that any arrangement that is calculated to achieve the same purposes can be substituted for the specific embodiments shown. This application is intended to cover any adaptations or variations of the embodiments and disclosure. For example, although described in terminology and terms common to the field of art, exemplary embodiments, systems, methods, and apparatus described herein, one of ordinary skill in the art will appreciate that implementations can be made for other fields of art, systems, apparatus, or methods that provide the required functions. The invention should therefore not be limited by the above-described embodiments, methods, and examples, but by all embodiments and methods within the scope and spirit of the invention.

In particular, one of the ordinary skills in the art will readily appreciate that the names of the methods and apparatus are not intended to limit embodiments or disclosures. Furthermore, additional methods, steps, and apparatus can be added to the components, functions can be rearranged among the components, and new components to correspond to future enhancements and physical devices used in embodiments can be introduced without departing from the scope of embodiments and the disclosure. One of the skills in the art will readily recognize that embodiments apply to future systems, future apparatus, future methods, and different materials.

All methods described herein can be performed in a suitable order unless otherwise indicated herein or otherwise clearly contradicted by context. The use of any, and all examples, or exemplary language (e.g., “such as”), is intended merely to better illustrate the disclosure and does not pose a limitation on the scope of the disclosure unless otherwise claimed. No language in the specification should be construed as indicating any non-claimed element as essential to the practice of the disclosure as used herein. The terminology used in the present disclosure is intended to include all environments and alternate technologies that provide the same functionality described herein.

1. A method for predicting wine preferences of a subject, the method comprising:
first determining phenotypic expression of T2Rs and/or T1Rs, wherein the determining is from a test comprising a plurality of agonists administered to detect the functionality of T2Rs and/or T1Rs;
obtaining taste preference information from the subject;
second, determining the subject's taste preference scores for one or more wines administered to the subject;
predicting wine preferences for the subject.

2. The method of claim 1, further comprising: calculating wine bin scores based on the first determining phenotypic expression, the obtaining taste preferences information, and the second determining the subject's taste preference scores.

3. (canceled)

4. (canceled)

5. (canceled)

6. (canceled)

7. (canceled)

8. (canceled)

9. (canceled)

10. (canceled)

11. (canceled)

12. The test method of claim 1, further comprising:
wherein the agonist(s) are selected from caffeine, denatonium, strychnine, quinine, terpenes, phenylthiocarbamide, thiourea, sodium benzoate, and any two or more of the foregoing, and
wherein the detecting of the products comprises applying in series a test medium containing each of the reagents which interact with the products.

13. (canceled)

14. The method of claim 1, wherein the first determining further comprises:
i) stimulating T2Rs and/or T1Rs by exposing at least a portion of mouth tissue of the human subject to one or more agonists,
ii) recording a discerned level of taste perception by the human subject after the stimulation, and
iii) correlating the discerned level of taste perception to the level of phenotypic expression of the T2Rs and/or T1Rs.

15. The method according to claim 14, further comprising: wherein the agonists are selected from the group consisting of caffeine, denatonium, strychnine, quinine, terpenes, phenylthiocarbamide, thiourea, sodium benzoate, and any two or more of the foregoing.

16. The method of claim 14, further comprising: wherein the first determining further comprises wherein the agonist is selected from the group consisting of caffeine, denatonium, strychnine, quinine, xylitol, grapefruit seed extract or naringenin, a terpene, and any two or more of the foregoing.

17. A method according to claim 14, further comprising: wherein:
steps i) and ii) are repeated one or more times, and the stimulating by each of one or more different agonists is sequential,
the recording of each discerned level of taste perception by the human subject occurs after each stimulation, and
the correlating is of one or more of the discerned levels of taste perception to the level of phenotypic expression of the T2Rs and/or T1Rs.

18. (canceled)

19. A method for predicting wine preferences of a subject, the method comprising:
first determining phenotypic expression of T2Rs and/or T1 Rs, wherein the determining is from an actual test administered to detect the functionality of T2Rs and/or T1Rs;
second determining the subject's taste preference scores for one or more wines administered to the subject;
predicting wine preferences for the subject.

20. The method of claim 19, further comprising:
calculating wine bin scores based on the first determining phenotypic expression, and the second determining the subject's taste preference scores.

21. (canceled)

22. (canceled)

23. (canceled)

24. (canceled)

25. The method of claim 19, further comprising:
wherein the agonists are separately applied prior to the reagent which interacts with one or more of the products as a result of stimulation of T2Rs and/or T1Rs.

26.-58. (canceled)

59. The method according to claim 14, said method further comprising: using a test kit configured to enable performing said method, said test kit comprising:
at least one of said agonists contained in at least one test medium.

60. The method according to claim 19, said method further comprising:
using a test kit configured to enable performing said method, said test kit comprising:
at least one of said agonists contained in at least one test medium.

61. The method according to claim 19, said method further comprising:
providing a computing platform that enables the execution of the method, the computing platform configured to recommend wines by determining taste perception of an individual and determining wine preferences in relation to taste perception.

62. The method according to claim 19, said method further comprising:
the computing platform configured to recommend wine formulations in relation to determining taste perception of an individual by diagnostic tests to establish taster group and determining wine preferences in relation to taste perception.

63. (canceled)

64. The method according to claim 19, said method further comprising:
the computing platform configured to provide wine formulation information in relation to taste perception of consumers, determined in relation to diagnostic tests establishing taster group and preferences in relation to taste perception.
